(19) United States
(12) Patent Application Publication
(10) Pub. No.: US 2026/0020829 A1
(43) Pub. Date: Jan. 22, 2026

(54) WHEELCHAIR HAVING AN IMAGE CASSETTE HOLDER

(71) Applicant: Charles Edward Porter, Fayetteville, NC (US)

(72) Inventor: Charles Edward Porter, Fayetteville, NC (US)

(21) Appl. No.: 18/777,449

(22) Filed: Jul. 18, 2024

(51) Int. Cl. A61B 6/04; A61G 5/00; A61G 5/12

(52) U.S. Cl. CPC A61B 6/0407; A61G 5/006; A61G 5/127; A61G 5/128; A61G 2210/50

(57) ABSTRACT

A wheelchair for radiographic testing is provided. An implementation of the wheelchair includes a frame made of a radiographically transmissive material, a seat connected to the frame, a backrest connected to the frame, a lower support connected to the frame, a plurality of wheels connected to the frame, the plurality of wheels for contacting the ground and at least one of the seat, the backrest, or the lower support for receiving an imaging plate while a person is seated in the wheelchair.

The present invention relates to wheelchairs. Wheelchairs may be used to transport individuals when walking is painful, difficult, or impossible, due to illness, injury, disability or age, for example.

### BACKGROUND

Wheelchairs typically include a metal frame with wheels mounted to the bottom of the frame. A seat and a seat back are supported by the frame. When non-ambulatory patients need radiographic testing, for example, X-rays, magnetic resonance imaging (MRI), or computed tomography (CT) scans, the patients are moved to another chair or bed suitable for use with the desired radiographic devices.

U.S. Pat. No. 4,831,644 discloses a modified wheelchair with detachable armrests and backrests, and a radiograph accessory consisting of a radiograph film cassette holder and an opposed brace member attachable to the modified wheelchair utilizing the same mounting used by the detachable armrests and backrests.

U.S. Pat. No. 7,597,339 discloses a wheelchair made of x-ray transmissive material for use with a radiographic device.

### SUMMARY

When non-ambulatory patients need radiographic testing services, patients are transported to radiology locations having radiographic equipment. The patients are then transferred from their personal wheelchair to a radiograph table or positioned near a wall-mounted radiograph machine. Lab technicians or assistants are often needed to help transfer the non-ambulatory patient from their wheelchair to the radiograph table and/or to keep the patient properly positioned during testing. The transfer and positioning during the testing procedures may cause the patients a great deal of pain or discomfort and may be physically strenuous, cumbersome or difficult for the lab technicians or assistants. In addition, the lab technicians or assistants may be subject to unnecessary radiation during patient testing.

Implementations of a wheelchair for radiographic testing may include a frame made of a radiographically transmissive material; a seat connected to the frame; a backrest connected to the frame; a lower support connected to the frame; a plurality of wheels connected to the frame, the plurality of wheels configured to contact a ground; and at least one of the seat, the backrest, or the lower support configured to receive an imaging plate while a person is seated in the wheelchair.

Further implementations of a wheelchair for radiographic testing include a radiographically transmissive frame; a seat mounted to the radiographically transmissive frame; a backrest mounted to the radiographically transmissive frame; a lower support connected to the radiographically transmissive frame; a plurality of wheels connected to the radiographically transmissive frame, the plurality of wheels configured to contact a ground; and at least one of the seat, the backrest, or the lower support having a holder for receiving an imaging plate.

The foregoing and other aspects, features, and advantages will be apparent to those artisans of ordinary skill in the art from the DESCRIPTION and DRAWINGS, and from the CLAIMS.

### BRIEF DESCRIPTION OF THE DRAWINGS

The present invention will be elucidated with reference to the following drawings, in which:

FIG. 1 is a perspective view of a wheelchair according to an implementation of the present invention;

FIGS. 2A to 2F illustrate a plurality of views of a wheelchair according to implementations of the present invention;

FIGS. 3A and 3B illustrate back views of a backrest and seat according to implementations of the present invention; and

FIGS. 4A to 4C illustrate back views of a backrest according to implementations of the present invention.

### DESCRIPTION

This disclosure, its aspects and implementations, are not limited to the specific components, assembly procedures or method elements disclosed herein. Many additional components, assembly procedures and/or method elements known in the art consistent with the intended wheelchair will become apparent for use with particular implementations from this disclosure. Accordingly, for example, although particular implementations are disclosed, such implementations and implementing components may comprise any shape, size, style, type, model, version, measurement, concentration, material, quantity, method element, step, and/or the like as is known in the art for such wheelchair, and implementing components and methods, consistent with the intended operation and methods.

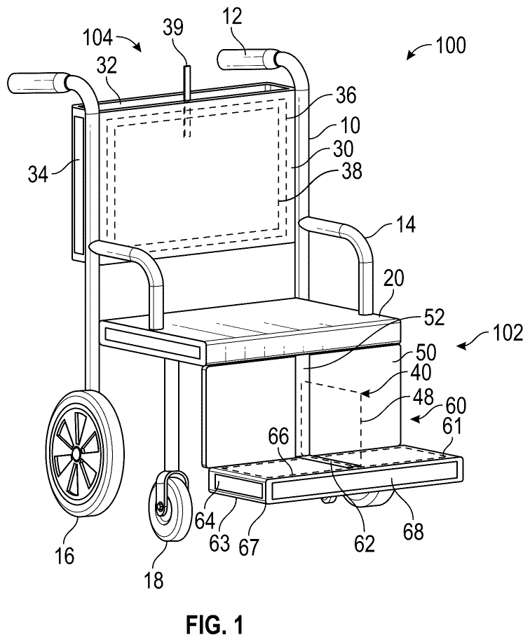

FIG. 1 is a perspective view of a wheelchair according to an implementation of the present invention. Wheelchair 100 allows patients, particularly non-ambulatory patients, to rest comfortably during radiographic testing of, for example, the patient's back, lungs, ribs and lower extremities including the patients' femur, knees, feet, ankles, tibia and fibula by providing holders, including receptacles, supports and couplers, that hold imaging plates or imaging cassettes in place during testing. In this manner, wheelchair 100 is configured to receive imaging plates or cassettes for use with radiographic testing equipment such that wheelchair 100 can be used with radiographic testing equipment instead of requiring patients to switch to a different chair or table or be near a wall mounted radiograph machine.

Wheelchair 100 includes a frame 10 having handles 12 and armrests 14. A seat 20, a backrest 30, and a lower support 40 are mounted to frame 10. Each of seat 20, backrest 30 and lower support 40 may be detachable from frame 10, for example, for cleaning, storage, replacement, etc. Wheels 16, 18 are also removably connected to frame 10. Wheels may be of varying sizes, for example, wheels 16 may be sized for manual control and wheels 18 may be smaller than wheels 16. Frame 10, seat 20, backrest 30, and lower support 40 may be made of a radiolucent material or a radiographically transmissive material and include, for example, carbon, plastic, fabrics, textiles, or polymers. Using a radiolucent or a radiographically transmissive material allows the radiation to pass through frame 10, seat 20, backrest 30, and lower support 40 to obtain a clear radiograph of the patient when an image cassette is coupled to a component of wheelchair 100.

In accordance with an implementation of wheelchair 100, seat 20, backrest 30 and/or lower support 40 include image cassette holders. In one implementation backrest 30 includes a central opening 36 for receiving an image cassette 38 therein. As shown in FIG. 1, backrest 30 has a top slot 32 on a top surface and a side slot 34 on a side surface thereof. Both top slot 32 and side slot 34 connect to central opening 36. Top slot 32, side slot 34 and central opening 36 are each sized to accommodate image cassette 38 so image cassette 38 can be placed into central opening 36 via top slot 32 or side slot 34. Thus, a lab technician or assistant can place an image cassette 38 into backrest 30 via top slot 32 or side slot 34. Advantageously, the lab technician or assistant may place image cassette 38 into central opening 36 while a patient is seated in wheelchair 100. Image cassette 38 may be placed into and removed from central opening 36 by manually pushing and pulling image cassette 38 into and out of central opening 36. In some implementations, a tail 39 or ribbon may be connected to image cassette 38 to ease the removal process. In other implementations, tail 39 or ribbon may also be used to fasten a free end of tail 39 to wheelchair 100 in order to arrange image cassette 38 at a desired height with respect to a seated patient. In some implementations backrest 30 has one of top slot 32 or side slot 34.

Lower support 40 is also configured to receive image cassettes. In some implementations, lower support 40 includes a leg rest 50 and a footrest 60. Leg rest 50 and footrest 60 may be rotatable with respect to each other and with respect to frame 10. Lower support 40 may be removable from frame 10 and footrest 60 may be removable from leg rest 50. Footrest 60 may be connected to frame 10 directly or indirectly and may be connected to frame 10 via leg rest 50.

As shown in FIG. 1, footrest 60 may be generally perpendicular to leg rest 50 or positioned at an ergonomic angle. Leg support 50 includes a groove, slot or opening 52 in which an image cassette 48 may be arranged. Slot 52 extends vertically and may be centrally located with respect to a width of leg rest 50 such that the slot 52 is arranged between a patient's right and left legs when a patient is seated in wheelchair 100. A height of slot 52 may be less than or equal to a height of leg rest 50. Slot 52 may extend through a full thickness of leg rest 50 from an anterior side 102 to a posterior side 104 defining an opening. Alternatively, slot 52 may extend into leg rest 50 a depth less than a thickness of leg rest 50 forming a groove. One end of image cassette 48 may be placed into slot 52 to position image cassette 48 between a patient's lower legs such that a radiograph may be taken of either leg from a right lateral side or a left lateral side of the patient.

Footrest 60 may also include a groove, slot or opening 62 in which image cassette 48 can be positioned. Preferably, slot 62 is aligned with slot 52, such that a bottom portion of image cassette 48 is received in slot 62 and a vertical side of image cassette 48 is received in slot 52. A lab technician or assistant may position an image cassette 48 into slots 52, 62 while a patient is seated in wheelchair 100. Allowing a patient to be radiographed seated in their wheelchair reduces the burden on the lab technicians to move non-ambulatory patients, reduces pain to the patient caused by forced and awkward movements and increases the ease and satisfaction of the patient's visit.

In one implementation footrest 60 includes a central opening 66 for receiving an image cassette therein. As shown in FIG. 1, footrest 60 has lateral slots 64 on lateral surfaces 61, 63 and a front slot 68 on a front surface 67 thereof. Both lateral slots 64 and front slot 68 connect to central opening 66 and are each sized to accommodate an image cassette so the image cassette can be placed into central opening 66 via front slot 68 or lateral slots 64. A lab technician or assistant can place an image cassette into footrest 60 via front slot 68 or lateral slots 64. Advantageously, the lab technician or assistant may place the image cassette into central opening 66 while a patient is seated in wheelchair 100. Image cassette may be placed into and removed from central opening 66 by manually pushing and pulling image cassette into and out of central opening 66. In some implementations, a tail or ribbon may be connected to the image cassette to ease the removal process. The tail or ribbon may also be used to position the image cassette laterally with respect to footrest 60.

In some implementations, lower support may be composed of right and left components adjacent one another and movable with respect to each other. A right side of the footrest and leg rest may move independent from a left side of the footrest and leg rest. In these implementations each right and left component of the footrest may include a central opening for receiving image cassettes.

By allowing image cassettes to be inserted and removed while a patient remains seated in wheelchair 100, transfer and positioning of the patient during testing procedures is reduced or eliminated which reduces the pain incurred by the patient during such transfers. In addition, lab technicians do not need physically exert themselves to transfer and position patients and can reduce their exposure to unnecessary radiation during patient testing because the patient is supported by the wheelchair.

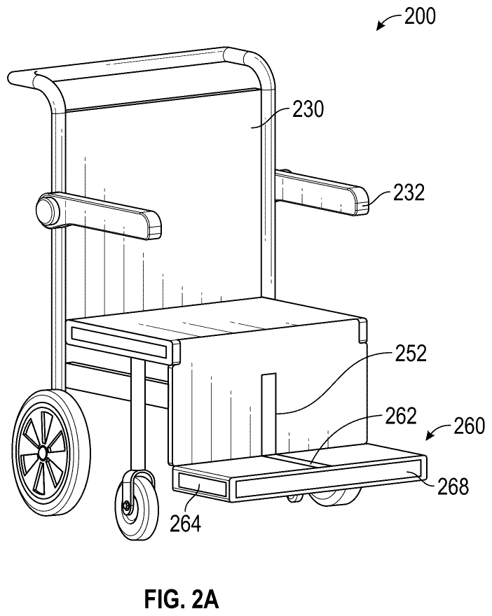

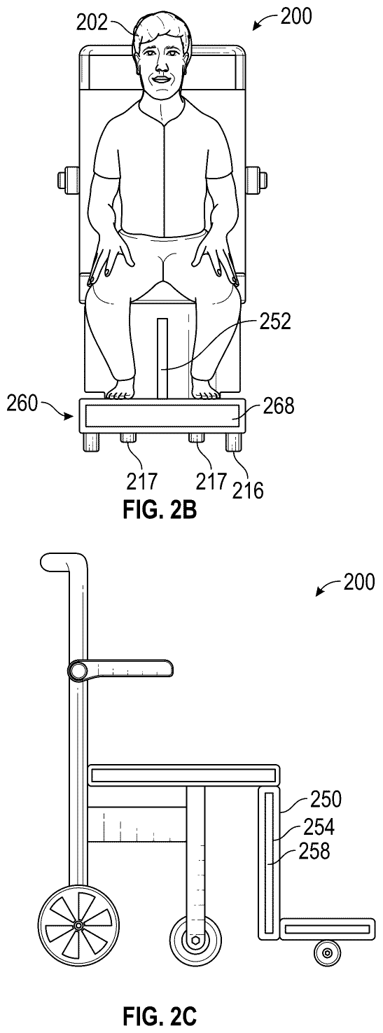

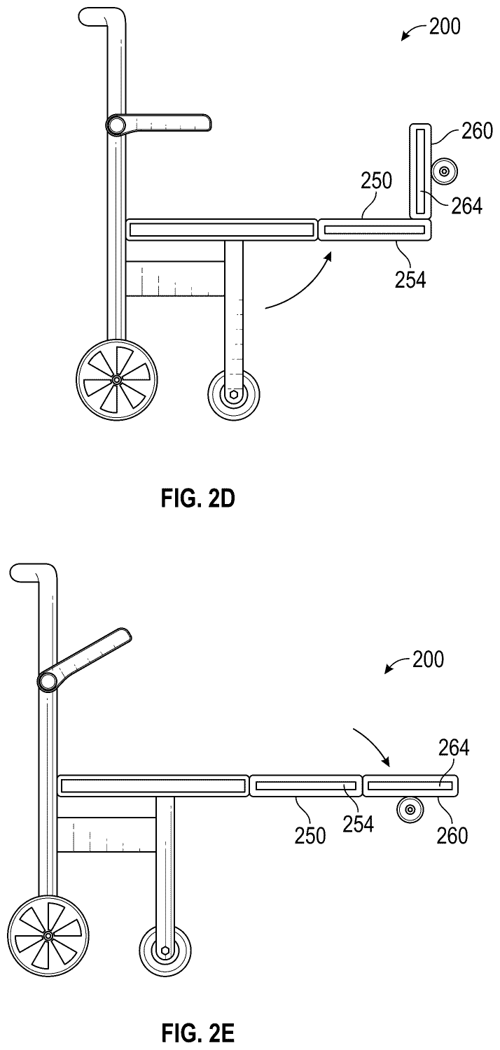

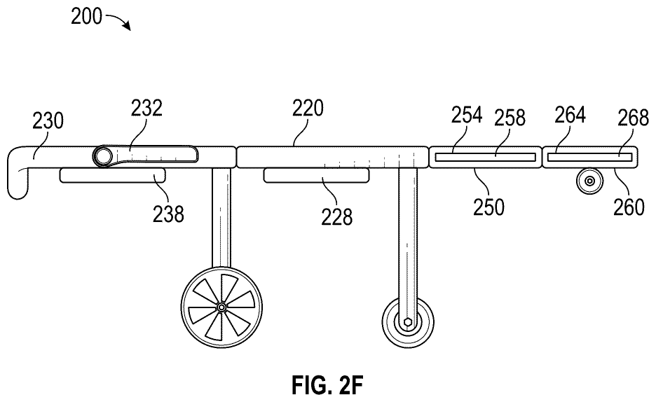

FIGS. 2A to 2F illustrate a wheelchair 200 in various positions. FIG. 2A illustrates a perspective view of wheelchair 200 showing a slot 252 in leg rest 250 and a slot 262 in footrest 260 for receiving an image cassette. FIG. 2B illustrates a front view of wheelchair 200 showing slot 252 and front slot 268 for receiving image cassettes. Wheels 217 connected to footrest 260 are also illustrated.

In FIGS. 2A and 2B, slots 252, 262 are configured to receive an image cassette positioned between the legs of a patient while a patient 202 is seated in wheelchair 200. Lateral slot 264 and front slot 268 in footrest 260 are also illustrated. An image cassette may be slid into slots 264 or 268 while a patient is seated in wheelchair 200 and/or is resting their feet on footrest 260. By providing an image cassette coupled to footrest 260, a patient can apply some pressure to their foot, for example, by leaning forward in wheelchair 260 or by pressing down on footrest 260. Applying pressure or weight, even minimal pressure or weight, to the patient's foot may increase the quality of the radiographic image obtained resulting in an improved diagnosis, specifically for middle foot fractures that often go undiagnosed. Thus, in these instances, a patient is not required to stand on his or her feet, bearing their body weight, which may be very painful. The patient can apply a lower amount of pressure to the foot while remaining seated in the wheelchair which improves the patient's testing experience and reduces pain. Advantageously, the image cassette is protected from damage or direct contact by the patient because the image cassette is inside footrest 260.

FIGS. 2C to 2F illustrate side views of wheelchair 200. FIG. 2C illustrates wheelchair 200 in a seated configuration. Leg rest 250 includes a slot 254 for receiving an image cassette 258 within a central opening therein. FIG. 2D illustrates wheelchair 200 with leg rest 250 in a raised position and footrest 260 perpendicular to leg rest 250. FIG. 2E further illustrates wheelchair 200 with leg rest 250 in the raised position and footrest 260 extended to align with leg rest 250. The configurations of wheelchair 200 in FIGS. 2D and 2E advantageously allow for radiography of a patient's knee from above while the patient is seated in wheelchair 100. The patient may remain seated in wheelchair 200 instead of being moved to a table or having to stand.

FIG. 2F illustrates a side view of wheelchair 200 in a fully extended position. Image cassette 238 is coupled to backrest 230, image cassette 228 is coupled to seat 220, image cassette 258 is inside slot 254 in leg rest 250 and image cassette 268 is inside slot 264 in footrest 260. A patient, initially seated in wheelchair 200, can be moved to a supine position by raising leg support 250 and extending footrest 260 to align with leg support 250 and by reclining backrest 230 to align with seat 220. An arm rest 232 may be rotated out of the way to align with backrest 230. A plurality of image cassettes may be coupled to different components of wheelchair 200 including seat 220, backrest 230, leg support 250 and footrest 260, to obtain desired radiographic images without moving the patient from wheelchair 200. Alternatively, a single image cassette may be reused and repositioned to capture the desired radiographic images. In various implementations, a digital image cassette can be reused after an image is transmitted to a computer, cloud, server, etc. In this manner, the lab technician can capture an image on the digital image cassette, reposition the cassette, and take a subsequent image until the desired images are captured, while the patient remains in wheelchair 200. For example, a digital image cassette may first be coupled to backrest 230, then coupled to seat 220 then inserted into slot 254 and then inserted into slot 264 to obtain various images of the patient while the patient is seated or reclined in wheelchair 200.

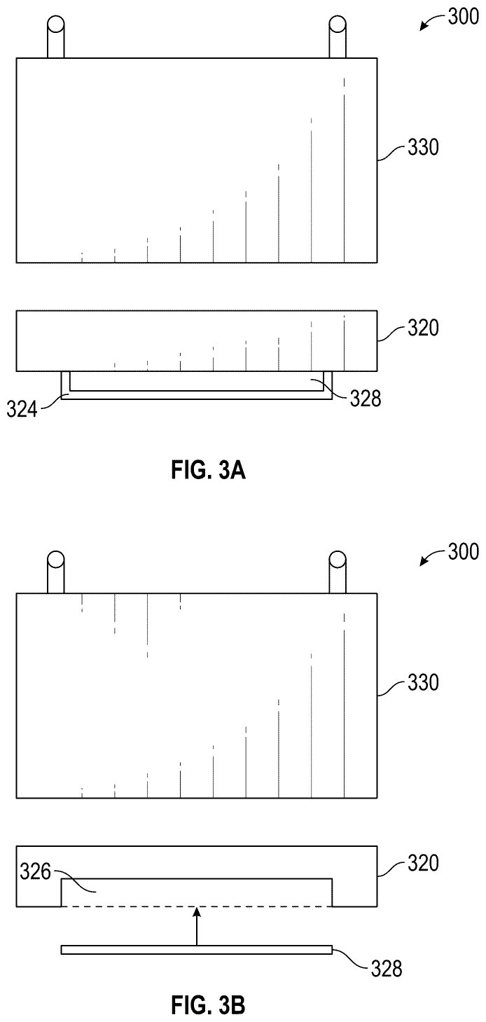

FIGS. 3A and 3B illustrate back views of backrest 330 and seat 320 which may be used with wheelchairs 300 according to various implementations of the present disclosure. Seat 320 may also be configured to support an image cassette 328. As shown in FIG. 3A, a holder or support 324 couples an image cassette 328 to a bottom side of seat 320. Support 324 may be, for example, a strap, a plurality of straps, a bracket or a pocket and may be connected to seat 320 on at least one end. A lab tech or assistant may position an image cassette 328 adjacent seat 320 via support 324 while a patient is seated in wheelchair 300 by sliding image cassette 328 in between support 324 and seat 320. In one implementation, support 324 is a pocket and image cassette 328 is slid into and out of pocket 324 as needed. In another implementation, support 324 includes at least one strap for supporting image cassette 328 in a position adjacent seat 320. In some implementations, the straps may be elastic, in other implementations the straps may have further coupling mechanisms such as snaps, hook and loop fasteners, buckles, etc., to facilitate supporting an image cassette 328 adjacent seat 320. In other implementations, elastic straps may be fixed to seat 320 and an image cassette 328 may be placed between the straps and seat 320. In still further implementations, a portion of a strap may be fixed to seat 320 and a free end of a strap may wrap around an image cassette to support the image cassette against seat 320. The free end of the strap may connect to the fixed portion of the strap, the seat or another strap by various coupling or fastening means. In another implementation, the seat and image cassette each include fastening or coupling devices for positioning the image cassette adjacent the seat. In one example, both the image cassette and seat may have hook and loop fasteners providing a simple yet sturdy fastening mechanism. A lab technician can align the hook and loop fasteners of the image cassette with corresponding fasteners on the seat and use gentle pressure to attach the image cassette to the wheelchair seat.

FIG. 3B illustrates another implementation of the present disclosure for coupling an image cassette 328 to a seat 320. Seat 320 includes a receptacle or depression 326 extending into a depth of seat 320 for receiving an image cassette 328 therein. The image cassette 328 may be secured in receptacle 326 by fasteners including clips and straps, for example.

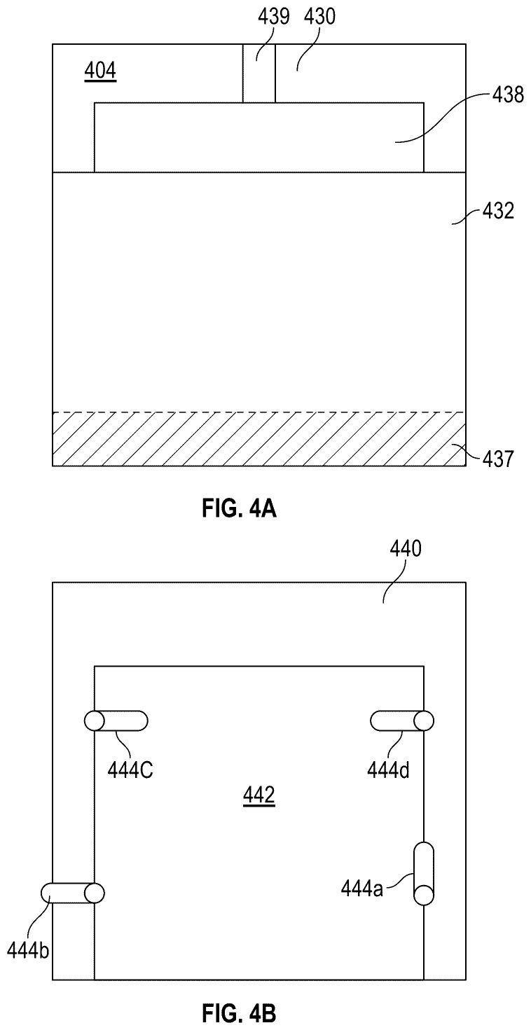

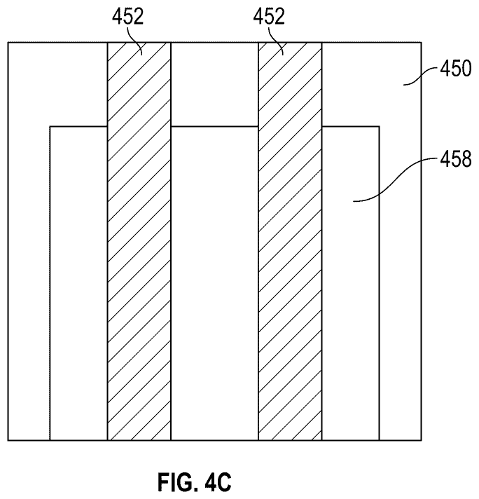

FIGS. 4A to 4C illustrate various implementations of coupling an image cassette to a wheelchair used during radiographic testing. While FIGS. 4A to 4C show a backrest, these implementations may also be used to couple an image cassette to a seat, lower support, leg rest and/or footrest of a wheelchair. FIG. 4A illustrates a pocket 432 on an anterior side 404 of backrest 430. An image cassette 438 can be slid into and out of pocket 432 easily while a patient is seated in the wheelchair. A tail or ribbon 439 on attached to image cassette 438 may be used to adjust the vertical placement of cassette 438 with respect to backrest 430. One end of tail 439 is connected to image cassette 438 while a second end of tail 439 may be free and/or connectable to backrest 430 or another accessible component or part of the wheelchair. In addition to or in lieu of tail 439 a bolster 437 may be placed in the bottom of pocket 432 to adjust a position of image cassette 438 with respect to backrest 430.

FIG. 4B illustrates a backrest 440 having a receptacle 442 that extends into a depth of backrest 440 to accommodate an image cassette therein. The image cassette may be secured in receptacle 442 by fasteners 444 of any type including clips and straps, for example. As illustrated in FIG. 4B, clips 444 are connected to backrest 440 and are rotatable. As shown, clips 444a, 444b are rotated away from receptacle 442 such that an image cassette can be placed into or removed from receptacle 442 whereas clips 444c, 444d are shown positioned over receptacle 442 to secure an image cassette in receptacle 442.

FIG. 4C illustrates a backrest 450 having straps 450 configured to support an image cassette 458 against backrest 450. Straps may be elastic, and/or removable, and/or include fasteners including snaps, buckles, hook and loop fasteners, etc. An image cassette 458 may be arranged between straps 452 and backrest 450 easily while a patient is seated in the wheelchair. In addition, a bolster or tail as illustrated in FIG. 4A may be used to adjust a vertical position of image cassette 458 with respect to backrest 450 if needed.

According to various implementations of the present disclosure, the imaging plate can be coupled to the wheelchair by clamps, groves, holes, adhesive tape, rope, straps, patient anatomy support, pouch, case, or bag.

In accordance with the present disclosure, image cassettes can be coupled to a wheelchair made of a radiolucent material or a radiographically transmissive material while a patient is seated in the wheelchair. Thus, the wheelchair allows for diagnostic images to be taken in a plurality of views including opposing views, which aids interpretation of the images. In addition, the images may be taken while a patient is seated in the wheelchair, reducing the amount of movement and reducing the opportunity for injury or pain to the patient, particularly for non-ambulatory patients. A wheelchair according to the present disclosure also allows images in a weight bearing position to be taken which display middle foot fractures that would normally go undiagnosed. For example, a patient, while seated, may lean forward and apply a gentle pressure to their foot sufficient for a successful image to be taken without having to stand or bear a significant amount of weight on their foot.

Another advantage of the present disclosure includes lowering severe pain imparted to the patient by limiting a patient's movement during testing. The patient will not have to be dangerously and uncomfortably moved from a chair to an examining table and the need for additional staff to help move the patient during the exam is also reduced. Multiple exposures to ionizing radiation to the patient can be reduced by using the wheelchair. More accurate and optimal images of the patient's anatomy may also be taken because the patient is resting more comfortably in the wheelchair. For example, diagnostic images in three opposing views may be taken. Images may be taken from above, below, right, left, front or back, for example.

The wheelchair also reduces the amount of time it takes to perform radiographic testing, allowing more patients to be seen in a timely manner. Increasing the number of patients seen in a day will also increase financial opportunities. The amount of time to see the next patient may decrease which can improve patient morale when waiting to be seen. Also, lab assistants and technicians are subject to less exposure of ionizing radiation as well as physical stress due to lifting and moving the patients. Various implementations of the wheelchair can help obtain diagnostic images for patients with impaired mobility, lower leg pain, children with special needs, seniors, as well as paraplegics. The wheelchair can be used in hospitals, ortho clinics, imaging clinics, urgent cares, hospices, nursing homes, mobile exams, or for personal use.

In places where the description above refers to particular implementations of a wheelchair and implementing components, sub-components, methods and sub-methods, it should be readily apparent that a number of modifications may be made without departing from the spirit thereof and that these implementations, implementing components, sub-components, methods and sub-methods may be applied to other wheelchairs.

What is claimed is:

1. A wheelchair for radiographic testing comprising:
a frame made of a radiographically transmissive material;
a seat connected to the frame;
a backrest connected to the frame;
a lower support connected to the frame;
a plurality of wheels connected to the frame, the plurality of wheels configured to contact a ground;
at least one of the seat, the backrest, or the lower support configured to receive an imaging plate while a person is seated in the wheelchair.

2. The wheelchair of claim 1, wherein the radiographically transmissive material of the frame is or includes carbon.

3. The wheelchair of claim 1, wherein the seat, the backrest and the lower support are radiographically transmissive.

4. The wheelchair of claim 1, wherein at least one of the seat, the backrest or the lower support are made of a radiographically transmissive material that includes a polymer, plastic, or fabric.

5. The wheelchair of claim 1, wherein at least one of the seat, the backrest, or the lower support includes a pocket, a slot or an opening for receiving the imaging plate.

6. The wheelchair of claim 1, wherein at least one of the seat, the backrest, or the lower support includes a coupler, a connector or a bracket for receiving the imaging plate.

7. The wheelchair of claim 6, wherein the coupler, the connector or the bracket includes snaps, a hook and loop fastener, clips, hooks, clamps, holes, adhesive tape, ropes, ties or straps.

8. The wheelchair of claim 1, wherein the lower support includes a leg rest and a footrest, the leg rest and footrest being rotatable with respect to each other and the leg rest and footrest each being rotatable with respect to the frame.

9. The wheelchair of claim 7, wherein the leg rest and footrest are each configured to receive an imaging plate.

10. The wheelchair of claim 1, wherein the lower support includes a leg rest and a footrest, the leg rest and footrest each having a slot or an opening configured to receive an imaging plate.

11. The wheelchair of claim 1, wherein the lower support includes a leg rest and a footrest and the lower support is detachable from the frame.

12. The wheelchair of claim 1, wherein the backrest, the seat and the lower support are configured to form a flat bed.

13. The wheelchair of claim 1, wherein the imaging plate can be moved so a plurality of diagnostic views may be recorded.

14. The wheelchair of claim 1, wherein imaging plates can be coupled to the wheelchair while a user is seated in the wheelchair.

15. The wheelchair of claim 1, wherein the seat, the backrest or the lower support are configured to house an imaging plate.

16. The wheelchair of claim 15, wherein the imaging plate is positioned between a front and a back or a top and a bottom of the seat, the backrest, or the lower support.

17. The wheelchair of claim 16, wherein the imaging plate is positioned in a depression, a receptacle or a slot.

18. The wheelchair of claim 1, wherein the footrest includes grooves or slots for receiving an imaging plate.

19. A wheelchair for radiographic testing comprising:
a radiographically transmissive frame;
a seat mounted to the radiographically transmissive frame;
a backrest mounted to the radiographically transmissive frame;
a lower support connected to the radiographically transmissive frame;
a plurality of wheels connected to the radiographically transmissive frame, the plurality of wheels configured to contact a ground;
wherein at least one of the seat, the backrest, or the lower support having a holder for receiving an imaging plate.

20. The wheelchair of claim 19, wherein the holder is an opening, slot, groove, pocket or fastener.
